(19) United States
(12) Patent Application Publication
(10) Pub. No.: US 2008/0242891 A1
(43) Pub. Date: Oct. 2, 2008

(54) Method for recovering cyclohexanone oxime

(75) Inventors: Masashi YOKOTA, Niihama-shi (JP); Kanji KUWAHARA, Niihama-shi (JP)

Correspondence Address: BIRCH STEWART KOLASCH & BIRCH, PO BOX 747, FALLS CHURCH, VA, 22040-0747, US

(73) Assignee: SUMITOMO CHEMICAL COMPANY, LIMITED

(21) Appl. No.: 12/078,278

(22) Filed: Mar. 28, 2008

(30) Foreign Application Priority Data
Mar. 29, 2007 (JP) 2007-087734
Dec. 19, 2007 (JP) 2007-327759

(51) Int. Cl. C07C 249/14

(52) U.S. Cl. 564/264

(57) ABSTRACT

A liquid cyclohexanone oxime A is vaporized by heating to obtain a cyclohexanone oxime gas A1 and the remaining residue C is distilled under a pressure lower than the pressure upon vaporization by heating, and then cyclohexanone oxime A2 contained in the residue C is recovered.

### BACKGROUND OF THE INVENTION

1. Field of the Invention

The present invention relates to a process for recovering cyclohexanone oxime contained in the residue remaining after vaporizing a liquid cyclohexanone oxime by heating.

2. Description of the Related Art

ε-caprolactam (hereinafter referred sometimes to as “lactam”) is a monomer which is mainly used to synthesize nylon-6 used in the production of fibers and engineering plastics. As a process for producing lactam, a process of carrying out a vapor-phase Beckmann rearrangement reaction using a gaseous cyclohexanone oxime as a starting material is known.

In the vapor-phase Beckmann rearrangement reaction, first, a liquid cyclohexanone oxime is usually evaporated by heating using a wetted-wall evaporator to obtain a gaseous cyclohexanone oxime (refer to US2002/0143181A1). However, cyclohexanone oxime may cause thermal decomposition or thermal condensation during the step of vaporizing by heating to produce tar as a by-product since it is inferior in thermostability. It is necessary to remove tar out of the system in the form of a residue since it causes clogging of the wetted-wall evaporator. In this case, there arises a problem that the liquid cyclohexanone oxime is also removed out of the system together with tar and thus gaseous cyclohexanone oxime cannot be obtained in high yield.

### SUMMARY OF THE INVENTION

Thus, an object of the present invention is to provide a process for efficiently recovering cyclohexanone oxime from the residue remaining after vaporizing the liquid cyclohexanone oxime by heating, using a simple and easy process, while taking account of the thermostability of cyclohexanone oxime.

Under these circumstances, the present invention provides a process for recovering cyclohexanone oxime, which comprises vaporizing a liquid cyclohexanone oxime by heating to obtain a cyclohexanone oxime gas (A1), distilling the remaining residue under a pressure lower than the pressure upon vaporization by heating, and recovering cyclohexanone oxime contained in the residue.

Furthermore, the present inventors have tested condensing a cyclohexanone oxime gas by a condenser so as to recover a cyclohexanone oxime gas separated by distillation under a low pressure in a liquid state in light of handiness in the case of reusing the recovered cyclohexanone oxime. However, they have found that when a cooling medium at a temperature of lower than 90° C. is used, a crystal of cyclohexanone oxime may be deposited in a condenser thereby causing clogging in the condenser since cyclohexanone oxime has a high melting point of 90° C. Furthermore, when hot water at a temperature of 90° C. or higher is used as a cooling medium of the condenser so as to prevent crystal deposition, the amount of uncondensed gas increases, resulting in non-negligible recovery loss since cyclohexanone oxime has a comparatively high vapor pressure. Also, crystal deposition in an effluent gas piping cannot be avoided, thereby causing clogging of the piping. As described above, in both cases, there was a problem that a continuous stable operation is obstructed by crystallization of cyclohexanone oxime. It is considered that the operation pressure upon distillation is controlled to normal pressure or higher so as to reduce the uncondensed gas when hot water at a temperature of 90° C. or higher is used as the cooling medium of the condenser. In that case, production of the tar component as the by-product is promoted. The present inventors have intensively studied so as to solve these problems, thus providing the following inventions.

(1) A process for recovering cyclohexanone oxime, which comprises vaporizing a liquid cyclohexanone oxime by heating to obtain a cyclohexanone oxime gas (A1), distilling the remaining residue under a pressure lower than the pressure upon vaporization by heating, and recovering cyclohexanone oxime contained in the residue.
(2) The process for recovering cyclohexanone oxime according to paragraph (1), wherein the distillation is carried out under a pressure of 1.4 to 10 kPa (absolute pressure).
(3) The process for recovering cyclohexanone oxime according to paragraph 1 or 2, wherein a cyclohexanone oxime gas (A2) separated by distilling the residue is brought into contact with a solvent capable of dissolving cyclohexanone oxime to obtain a cyclohexanone oxime solution, and then cyclohexanone oxime is recovered.
(4) The process for recovering cyclohexanone oxime according to paragraph 1 or 2, wherein the cyclohexanone oxime gas (A2) separated by distilling the residue is condensed by a condenser in which a cooling medium at 90° C. or higher circulates to obtain a cyclohexanone oxime condensate liquid (A3) and an uncondensed cyclohexanone oxime gas (A4) obtained simultaneously is brought into contact with a solvent capable of dissolving cyclohexanone oxime to obtain a cyclohexanone oxime solution, and then cyclohexanone oxime is recovered.
(5) The process for recovering cyclohexanone oxime according to paragraph 3 or 4, wherein the cyclohexanone oxime gas (A2) or the cyclohexanone oxime gas (A4) is brought into contact with the solvent on the surface of a cooling body.

The present invention exerts the effect of being capable of obtaining cyclohexanone oxime in a high recovery ratio, simply and easily, from a mixture of cyclohexanone oxime obtained as the residue after vaporizing the liquid cyclohexanone oxime and a tar component derived from cyclohexanone oxime. Moreover, according to the present invention, cyclohexanone oxime can be separated at a comparatively low temperature and also thermal degradation of the resulting cyclohexanone oxime can be suppressed since the residue is distilled under a low pressure. In addition, by bringing cyclohexanone oxime separated by distillation under a low pressure into contact with a solvent capable of dissolving cyclohexanone oxime to obtain a cyclohexanone oxime solution, it becomes possible to recover cyclohexanone oxime in a liquid state while suppressing agglomeration (crystal deposition) of cyclohexanone oxime in the condenser or the piping connected to the condenser, and thus handiness in the case of reusing the recovered cyclohexanone oxime can be improved.

By reusing the cyclohexanone oxime obtained by the present invention as a raw material for production of lactam through the vapor-phase Beckmann rearrangement reaction, the effect of being capable of establishing a production process of lactam which has high utilization efficiency of cyclohexanone oxime as a raw material and is excellent in productivity is also exerted.

### BRIEF DESCRIPTION OF THE DRAWINGS

FIG. 1 is a flow chart showing one embodiment of a process for recovering cyclohexanone oxime of the present invention.

FIG. 2 is a flow chart showing another embodiment of a process for recovering cyclohexanone oxime of the present invention.

FIG. 3 is a flow chart showing still another embodiment of a process for recovering cyclohexanone oxime of the present invention.

### DESCRIPTION OF REFERENCE NUMERALS

100: Evaporator
101: Vacuum distiller
102: First stage condenser
103: Gas-liquid contactor
104, 106: Pump (vacuum pump)
105: Cooling unit

### DETAILED DESCRIPTION OF THE INVENTION

The process for recovering cyclohexanone oxime of the present invention is a process for recovering cyclohexanone oxime from the residue remaining after vaporizing a liquid cyclohexanone oxime by heating so as to obtain a cyclohexanone oxime gas (A1) used as a raw material in the production of ε-caprolactam by a vapor-phase Beckmann rearrangement reaction. Specifically, the residue is a mixture of cyclohexanone oxime and a tar component composed of a thermally decomposed product of the cyclohexanone oxime, and the present invention pertains to a process for recovering cyclohexanone oxime by separating from the mixture.

Evaporation of the liquid cyclohexanone oxime by heating in the case of obtaining the residue is usually carried out in a state where the pressure is not reduced, namely, a state of normal pressure to an increased pressure. Specifically, evaporation is carried out by heating at a temperature of 130 to 170° C. under a pressure of 101 to 140 kPa (absolute pressure).

In the present invention, the residue is distilled under a pressure which is lower than the pressure upon vaporization by heating for obtaining a cyclohexanone oxime gas (A1) (hereinafter referred sometimes as to “Low Pressure”). Since cyclohexanone oxime can be separated at a comparatively low temperature by distilling under Low Pressure, thermal degradation of the resulting cyclohexanone oxime can be suppressed, and thus a high recovery ratio can be achieved. For example, the distillation is preferably carried out under reduced pressure of 1.4 to 10 kPa (absolute pressure), and more preferably 1.4 to 8 kPa (absolute pressure). When the pressure upon distillation is too low, cyclohexanone oxime tends to be agglomerated. In contrast, when the pressure upon distillation is too high, thermal degradation of cyclohexanone oxime tends to proceed.

The temperature (distillation temperature) upon distillation is preferably from 90 to 135° C., and more preferably from 90 to 125° C. When the distillation temperature is lower than 90° C., cyclohexanone oxime tends to be agglomerated. In contrast, when the distillation temperature is too high, thermal degradation of cyclohexanone oxime tends to proceed.

The cyclohexanone oxime gas (A2) separated by distilling the residue under Low Pressure is (I) recovered as a cyclohexanone oxime solution by bringing into contact with a solvent capable of dissolving cyclohexanone oxime, or (II) condensed by a condenser in which a cooling medium at a temperature of 90° C. or higher circulates to obtain a cyclohexanone oxime condensate liquid (A3), and then the uncondensed cyclohexanone oxime gas (A4) obtained simultaneously is preferably brought into contact with a solvent capable of dissolving cyclohexanone oxime to obtain a cyclohexanone oxime solution, which is then recovered. Consequently, handiness in the case of reusing the recovered cyclohexanone oxime can be improved while suppressing agglomeration (crystal deposition) of cyclohexanone oxime in the condenser or the piping connected thereto.

As the solvent, solvents capable of dissolving cyclohexanone oxime can be used. Specifically, alcohols such as methanol, ethanol, n-propanol, isopropanol, and t-butanol; toluene and cyclohexanone are preferably used.

When the cyclohexanone oxime gas (A2) or the cyclohexanone oxime gas (A4) is brought into contact with the solvent, either (i) a gas-liquid contactor which can bring the cyclohexanone oxime gas (A2) or (A4) as a vapor into contact with the solvent, or (ii) a condenser which can bring the cyclohexanone oxime gas (A2) or (A4) into contact with the solvent on the surface of a cooling body can be used. Specifically, the gas-liquid contactor (i) may be a contactor in which the solvent cooled to the temperature lower than a boiling point under an operation pressure of distillation under Low Pressure is sprayed in the vapor of the cyclohexanone oxime gas (A2) or (A4) using a spray nozzle and, for example, conventional gas absorption equipment such as a spray column and a packed column can be employed. In contrast, the condenser (ii) may be a condenser in which the solvent is sprayed over a condensate produced by cooling vapor of the cyclohexanone oxime gas (A2) or (A4) on the surface of a cooling body (heat exchanger, etc.) and, for example, conventional heat exchangers such as a multitubular heat exchanger can be employed.

By reusing the cyclohexanone oxime obtained by the present invention as a raw material for production of lactam through the vapor-phase Beckmann rearrangement reaction, it is possible to establish a production process of lactam which has high utilization efficiency of cyclohexanone oxime as a raw material and is excellent in productivity.

### EXAMPLES

The present invention will now be described in detail by way of Examples, but the present invention is not limited to the following Examples.

### Example 1

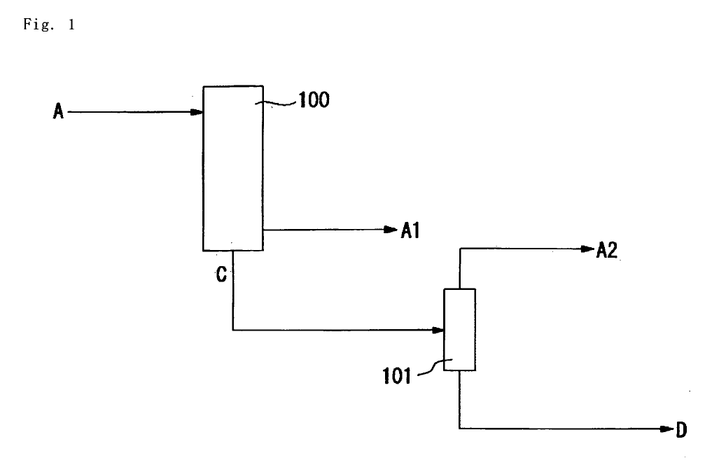

In accordance with the flow chart shown in FIG. 1, cyclohexanone oxime was recovered.

First, a liquid cyclohexanone oxime A was supplied to an evaporator 100 heated by steam (not shown) and then vaporized by heating under a pressure of 116 kPa (absolute pressure). As a result, a cyclohexanone oxime gas A1 was generated and also a mixture of cyclohexanone oxime and a tar component derived from cyclohexanone oxime was obtained as a residue C at a rate of 10 parts by weight/hour. The residue C was introduced into a vacuum distiller 101 heated by steam (not shown) and then distilled at 120° C. after evacuating the distiller 101 to 4.9 kPa (absolute pressure) using a vacuum pump (not shown). Then, the cyclohexanone oxime A2 was separated at a rate of 9 parts by weight/hour and also a tar component D as a distilled residue was discharged from the bottom of the vacuum distiller 101 at a rate of 1 part by weight/hour.

### Example 2

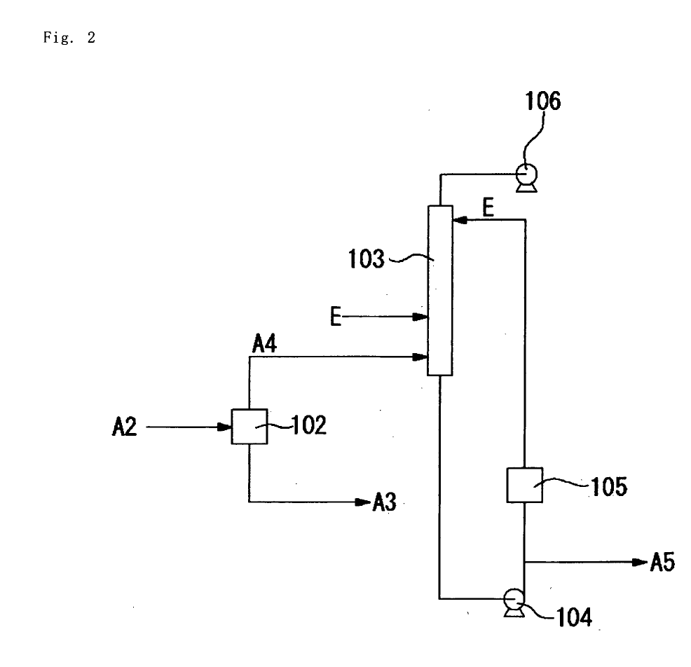

Using the cyclohexanone oxime A2 obtained by separating using the vacuum distiller 101 in Example 1, cyclohexanone oxime was recovered in accordance with the flow chart shown in FIG. 2.

The separated cyclohexanone oxime A2 was introduced into a first stage condenser 102 using hot water at 90° C. and a liquid cyclohexanone oxime A3 condensed in the first stage condenser 102 was recovered, and also an uncondensed cyclohexanone oxime gas A4 was introduced into a gas-liquid contactor 103 while maintaining at 95° C. The gas-liquid contactor 103 is a packed column in which methanol E introduced continuously is circulated by a pump 104 and is sprayed through a spray nozzle (not shown) from the upper portion of the gas-liquid contactor 103, and then the cyclohexanone oxime gas A4 is liquefied by bringing methanol E cooled to −6° C. using a cooling unit 105 directly into contact with the introduced cyclohexanone oxime gas A4 through spraying. Methanol containing the liquefied cyclohexanone oxime was extracted as a methanol solution A5 of cyclohexanone oxime during circulation. Cyclohexanone oximes A2 and A4 were introduced into the first stage condenser 102 and the gas-liquid contactor 103 using a vacuum pump 106.

### Example 3

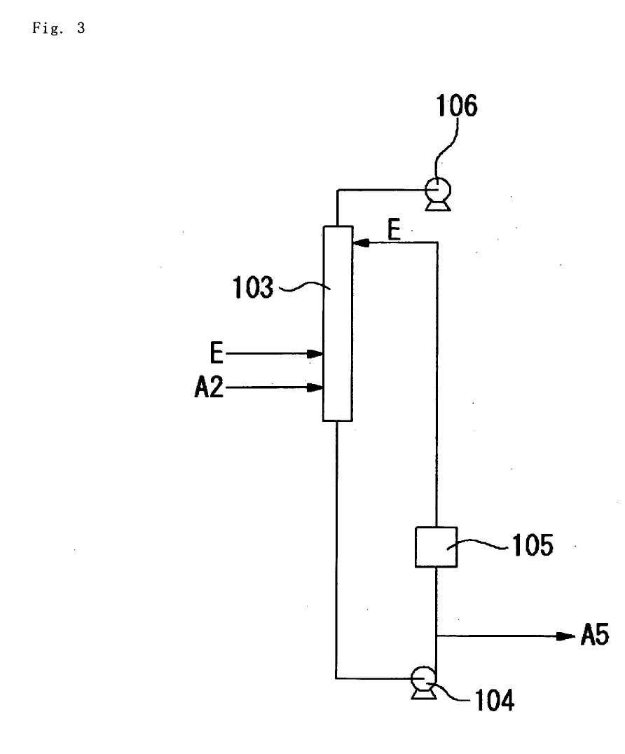

Using the cyclohexanone oxime A2 obtained by separating using the vacuum distiller 101 in Example 1, cyclohexanone oxime is recovered in accordance with the flow chart shown in FIG. 3.

The separated cyclohexanone oxime A2 was introduced into the gas-liquid contactor 103 using the vacuum pump 106. The gas-liquid contactor 103 is an equipment in which methanol E introduced continuously is circulated by the pump 104 and is sprayed through a spray nozzle (not shown) from the upper portion of the gas-liquid contactor 103, and then cyclohexanone oxime A2 is liquefied by bringing methanol E cooled to −6° C. using the cooling unit 105 directly into contact with the introduced cyclohexanone oxime gas A4 through spraying. Methanol containing the liquefied cyclohexanone oxime can be extracted as a methanol solution A5 of cyclohexanone oxime during circulation.

### INDUSTRIAL APPLICABILITY

The present invention exerts the effect of being capable of obtaining cyclohexanone oxime in a high recovery ratio, simply and easily, from a mixture of cyclohexanone oxime obtained as the residue after vaporizing the liquid cyclohexanone-oxime and a tar component derived from cyclohexanone oxime. Moreover, according to the present invention, cyclohexanone oxime can be separated at a comparatively low temperature and also thermal degradation of the resulting cyclohexanone oxime can be suppressed since the residue is distilled under Low Pressure. In addition, by bringing cyclohexanone oxime separated by distillation under Low Pressure into contact with a solvent capable of dissolving cyclohexanone oxime to obtain a cyclohexanone oxime solution, it becomes possible to recover cyclohexanone oxime in a liquid state while suppressing agglomeration (crystal deposition) of cyclohexanone oxime in the condenser or the piping connected to the condenser, and thus handiness in the case of reusing the recovered cyclohexanone oxime can be improved.

By reusing the cyclohexanone oxime obtained by the present invention as a raw material for production of lactam through the vapor-phase Beckmann rearrangement reaction, the effect of being capable of establishing the production process of lactam which has high utilization efficiency of cyclohexanone oxime as a raw material and is excellent in productivity is also exerted.

The present application has been filed claiming the Paris Convention priorities based on Japanese patent application No. 2007-087734 and Japanese patent application No. 2007-327759, the entire contents of which are herein incorporated by reference.

What is claimed is:

1. A process for recovering cyclohexanone oxime, which comprises vaporizing a liquid cyclohexanone oxime by heating to obtain a cyclohexanone oxime gas (A1), distilling the remaining residue under a pressure lower than the pressure upon vaporization by heating, and recovering cyclohexanone oxime contained in the residue.

2. The process for recovering cyclohexanone oxime according to claim 1, wherein the distillation is carried out under a pressure of 1.4 to 10 kPa (absolute pressure).

3. The process for recovering cyclohexanone oxime according to claim 1 or 2, wherein a cyclohexanone oxime gas (A2) separated by distilling the residue is brought into contact with a solvent capable of dissolving cyclohexanone oxime to obtain a cyclohexanone oxime solution, and then cyclohexanone oxime is recovered.

4. The process for recovering cyclohexanone oxime according to claim 1 or 2, wherein the cyclohexanone oxime gas (A2) separated by distilling the residue is condensed by a condenser in which a cooling medium at 90° C. or higher circulates to obtain a cyclohexanone oxime condensate liquid (A3) and an uncondensed cyclohexanone oxime gas (A4) obtained simultaneously is brought into contact with a solvent capable of dissolving cyclohexanone oxime to obtain a cyclohexanone oxime solution, and then cyclohexanone oxime is recovered.

5. The process for recovering cyclohexanone oxime according to claim 3, wherein the cyclohexanone oxime gas (A2) or the cyclohexanone oxime gas (A4) is brought into contact with the solvent on the surface of a cooling body.

6. The process for recovering cyclohexanone oxime according to claim 4, wherein the cyclohexanone oxime gas (A2) or the cyclohexanone oxime gas (A4) is brought into contact with the solvent on the surface of a cooling body.
